(19) United States
(12) Patent Application Publication
(10) Pub. No.: US 2017/0173268 A1
(43) Pub. Date: Jun. 22, 2017

(54) NUT ASSEMBLY FOR A MEDICAL DEVICE

(71) Applicant: Novo Nordisk A/S, Bagsvaerd (DK)

(72) Inventor: Christian Peter Enggaard, Vejby (DK)

(21) Appl. No.: 15/327,284

(22) PCT Filed: Jul. 10, 2015
(86) PCT No.: PCT/EP2015/065854

§ 371 Date: Jan. 18, 2017

(30) Foreign Application Priority Data
Jul. 25, 2014 (EP) 14178575.8

(51) Int. Cl. A61M 5/315; A61M 5/32; A61M 5/20

(52) U.S. Cl. CPC A61M 5/31536; A61M 5/20; A61M 5/31583; A61M 5/3202; A61M 5/31553

(57) ABSTRACT

The present invention provides a medical device (200) comprising a housing (201, 202), a guide structure (205) arranged stationarily with respect to the housing (201, 202) and ex-tending along a longitudinal axis, the guide structure (205) comprising a helical track (287) and a longitudinal track (289) being connected with the helical track (287) at a transition point (288), and a nut assembly (220) adapted to move along the guide structure (205). The nut assembly (220) comprises a first nut member (221) arranged in engagement with the helical track (287), a second nut member (321) capable of engagement with the helical track (287) and with the longitudinal track (289), the second nut member (321) being rotationally locked to the first nut member (221), and a bias member (296) arranged to bias the first nut member (221) and the second nut member (321) away from one another, such that the first nut member (221) and the second nut member (321) take up a first relative axial position when they are both in engagement with the helical track (287) and a second relative axial position when the second nut member (321) is in engagement with the longitudinal track (289).

### FIELD OF THE INVENTION

The present invention relates generally to locking arrangements for locking two components relative to one another, and more specifically to such locking arrangements suitable for use in medical devices, such as e.g. automatic injection devices.

### BACKGROUND OF THE INVENTION

Many devices depend on an ability to reliably position two components relative to one another to provide a stable state. In the medical device area, for example, a spring-driven injection device may need a secure storing of a loaded amount of energy in relation to a certain set dose, and thus the spring must be safely locked in the strained state until the user decides to release the energy for injection.

As a specific example hereof WO 2009/092807 (Novo Nordisk A/S) discloses an injection device where a torsionally pre-strained compression spring is used to securely position a piston rod driver on a shelf during a cap induced dose setting. When the cap is removed before a subsequent injection the compression spring remains stably cocked until the user pushes an injection button and thereby provides a torque which forces the piston rod driver over the edge of the shelf against the torsional bias of the spring to release the energy stored in the spring for translational advancement of the piston rod driver.

The solution disclosed in WO 2009/092807 is designed for an injection device where the spring loaded piston rod driver travels a certain distance axially during dose setting and the same distance in the opposite direction during the subsequent dose administration. However, it would be desirable to offer a similarly reliable solution for a device where a spring loaded element does not necessarily undergo such pattern of movement during straining and subsequent relaxation of the spring, to thereby provide for a greater flexibility of the device.

### SUMMARY OF THE INVENTION

It is an object of the invention to eliminate or reduce at least one drawback of the prior art, or to provide a useful alternative to prior art solutions.

In particular, it is an object of the invention to provide a device where two components may be stably positioned relative to one another in a simple and reliable manner.

It is a further object of the invention to provide a medical device where two components may be stably positioned relative to one another at a predetermined site following a non-predetermined relative motion.

In the disclosure of the present invention, aspects and embodiments will be described which will address one or more of the above objects and/or which will address objects apparent from the following text.

In one aspect of the invention a locking arrangement is provided comprising a) a guide structure extending along a longitudinal axis and comprising a1) a helical track, and a2) a longitudinal track being connected with the helical track at a transition point, and b) a nut assembly for moving along the guide structure, the nut assembly comprising b1) a first nut member arranged in engagement with the helical track, and b2) a second nut member capable of engagement with the helical track, respectively with the longitudinal track. The second nut member is rotationally locked with respect to the first nut member and biased axially away from the first nut member, such that when the second nut member is in engagement with the helical track the first nut member and the second nut member are axially spaced apart a first distance, and when the second nut member is in engagement with the longitudinal track the first nut member and the second nut member are axially spaced apart a second distance which is greater than the first distance.

A locking arrangement as the above described enables an automatic locking of the nut assembly with respect to the guide structure in response to the second nut member reaching a particular position. So long as both the first nut member and the second nut member are in engagement with the helical track they are prevented from undergoing relative axial motion and are thus axially locked with respect to one another. Since they are also rotationally locked with respect to one another they will travel the helical path in unison. However, when the second nut member reaches the transition point the axial bias forces the second nut member a distance axially along the longitudinal track while the first nut member remains in the helical track. Thereby, the nut assembly is effectively immobilised on the guide structure because the second nut member is prevented from rotating in the longitudinal track and the rotational coupling between the first nut member and the second nut member thus also prevents the first nut member from undergoing any helical movement.

The axial movement of the second nut member may be reversible so as to provide for a releasable locking of the nut assembly. For example, the second nut member may be movable along the longitudinal track towards the transition point by a dedicated user action such as a depression of a button or position of a cap. When the second nut member then reaches the transition point a translational or rotational force, e.g. applied by a spring, may lead it back into the helical track and move the entire nut assembly back along the helical track. This provides for a repetitive locking and release of the nut assembly relative to the guide structure. The locking arrangement is in principle suitable for all technical applications where an automatic locking in position of a first component relative to a second component is desired. It can for example be utilised for cocking of a spring or for secure coupling or decoupling of interacting components, and it is particularly useful within medical devices which will be clear from the below.

Hence, in another aspect of the invention a medical device is provided comprising a housing, a guide structure arranged stationarily with respect to the housing and extending along a longitudinal axis, and a nut assembly adapted to move along the guide structure. The guide structure comprises a helical track and a longitudinal track which is connected with the helical track at a transition point. The nut assembly comprises a first nut member arranged in engagement with the helical track, a second nut member structured for engagement with the helical track, respectively with the longitudinal track, and a bias member arranged to bias the first nut member and the second nut member axially away from one another. The first nut member and the second nut member are rotationally interlocked and thus unable to undergo any relative angular displacement about the longitudinal axis.

For engagement with the helical track the first nut member may comprise a radially inwardly protruding structure, such as a helical thread segment or a stub member, and for engagement with the helical track and with the longitudinal track the second nut member may comprise a track follower, such as e.g. a radially inwardly protruding stub member.

In a situation where both the first nut member and the second nut member are in engagement with the helical track they take up a first relative axial position which is invariable due to their rotational relationship. The nut assembly thus travels the helical track as one unitary element. However, when the second nut member reaches the transition point the bias provided by the bias member will displace the second nut member along a portion of the longitudinal track, whereby the nut assembly becomes effectively prevented from further rotation and is immobilised on the guide structure in a state where the first nut member and the second nut member take up a second relative axial position. In the second relative axial position the axial distance between the first nut member and the second nut member is greater than in the first relative axial position. The axial distance between the first nut member and the second nut member in their second relative axial position may be predetermined, e.g. by the choice of bias member.

The automatic locking in position of the nut assembly relative to the guide structure enables, for example, the provision of a simple and reliable spring cocking mechanism. Some medical devices employ a spring element to execute, or assist in the execution of, a particular action. Injection devices may e.g. use a compression spring or a torsion spring to provide energy for the pressurisation of a liquid carrying container, blood sampling devices may e.g. use a spring to propel a retractable lancet through an aperture, inhalation devices may e.g. use a spring to compress a canister against a nozzle, and intranasal dispensers may e.g. use a spring to displace a liquid carrying container towards a penetration element. During the lifetime of such devices it is often necessary to cock the incorporated spring element a number of times to allow repetition of the particular action.

The medical device may further comprise a spring, and a spring interfacing structure interacting with both the spring and the nut assembly. The spring interfacing structure may be adapted to tension the spring when the nut assembly travels the helical track towards the transition point. Thereby, when the second nut member reaches the transition point and is pushed away from the first nut member the tensioned spring is securely cocked as the nut assembly is stably immobilised on the guide structure. Specifically, the spring may be arranged to act between a portion of the housing and the spring interfacing structure, and the spring interfacing structure may be axially fixed with respect to the first nut member.

The spring may e.g. be a linear spring, such as a compression spring, in which case the helical track is non-self-locking to allow a helical displacement of the nut assembly in response to an axial load on the spring interfacing structure. The spring may alternatively be a torsion spring which may e.g. be tensioned by a rotational movement of the spring interfacing structure. In that case the helical track may be self-locking.

The displacement of the second nut member along the longitudinal track may be reversible to allow for a release of the cocked spring. The second nut member may be moved back to the transition point by a user pushing the second nut member along the longitudinal track with her/his finger against the bias of the bias member. Alternatively, the medical device may further comprise a nut release structure comprising a nut return button displaceable relative to the housing from a first position to a second position, where the nut release structure is coupled with the second nut member and configured to cause movement of the second nut member along the longitudinal track to the transition point against the bias of the bias member in response to a displacement of the nut return button from the first position to the second position.

When the second nut member reaches the transition point it automatically re-enters into engagement with the helical track due to the force (or torque) from the tensioned spring acting on the spring interfacing structure, and the following relaxation of the spring causes the nut assembly to move back along the helical track. Hence, as long as the second nut member is in engagement with the longitudinal track, the spring is securely cocked and the nut member is immobilised on the guide structure, but when the second nut member is moved to the transition point the spring is released and urged to deliver the stored energy.

The nut assembly may be displaceable along the helical track by the user, e.g. via an interaction with the spring interfacing structure or by use of a separate dedicated tool. In particular, the medical device may further comprise a removably mountable cap, such as a protective cap for covering a specific portion of the medical device when the medical device is not in use, and a cap receiving portion for accommodating a portion of the cap when the cap is mounted on the housing.

The cap may be used to displace the nut assembly along the helical track during mounting of the cap onto the housing. For example, the cap may be configured to interact directly with the nut assembly, e.g. via engagement means on the first nut member arranged to receive a portion of the cap in a rotational interlocking engagement. Alternatively, the cap may be configured to interact with the nut assembly via the spring interfacing structure, e.g. such that when the cap is being mounted onto the housing an edge of the cap abuts the spring interfacing structure and moves the spring interfacing structure along the longitudinal axis.

Regardless of which, by enabling operation of the nut assembly via the cap receiving portion a simple and user-friendly arrangement is provided since the user may then automatically bring the nut assembly to the immobilised position on the guide structure when mounting the cap onto the housing. If the cap is adapted to be mounted on the housing in a non-use state of the medical device the step of immobilising the nut assembly on the guide structure, e.g. for cocking the spring, is carried out automatically as part of a handling step which the user performs anyway during normal handling of the medical device, and a separate immobilisation step may therefore be spared.

The automatic locking in position of the nut assembly relative to the guide structure may alternatively, or additionally, enable an automatic coupling and/or decoupling of components. For example, the nut release structure may further comprise a nut connector being axially locked with respect to the second nut member and further rotationally locked with respect to the first nut member as long as the first nut member and the second nut member take up the first relative position. The nut connector is thus rotationally coupled with the first nut member as long as the second nut member is in engagement with the helical track. However, when the second nut member reaches the transition point and is moved away from the first nut member by the bias member, the nut connector undergoes a relative axial movement with respect to the second nut member which may cause the nut connector to become rotationally decoupled from the second nut member.

Alternatively, or additionally, the axial movement of the nut connector caused by the axial movement of the second nut member along the longitudinal track may cause the nut connector to move into engagement with another component in the medical device, as will be clear from the below description of an exemplary embodiment of the invention. It is thus clear that the present invention provides advantages irrespective of whether the medical device is a spring based device.

In particular embodiments of the invention the medical device is a drug delivery device, such as e.g. an injection device. In some such embodiments the medical device thus further comprises a variable volume reservoir being adapted to hold a liquid substance and having an outlet sealed by a penetrable septum. In other such embodiments the medical device further comprises means for receiving such a reservoir.

Regardless, the drug delivery device may further comprise a dose setting mechanism operable to set a dose to be delivered from the variable volume reservoir, or a received variable volume reservoir, and the dose setting mechanism may comprise a scale drum with respect to which the nut connector is rotationally locked. The scale drum may be in threaded engagement with an interior wall of the housing and thus capable of helical displacement inside a portion of the housing. The scale drum comprises a scale defining a range of settable doses and is configured to be positioned relative to the housing in accordance with the setting of a dose. The drug delivery device may further comprise a dose delivery mechanism adapted to deliver a set dose upon activation by a user, whereby the scale drum may be caused to move into abutment with a halting geometry.

The rotational interlocking connection between the scale drum and the nut connector entails a rotational interlocking connection between the scale drum and the first nut member when the first nut member and the second nut member take up the first relative position. Thereby, the scale drum and the first nut member are forced to rotate together as long as the second nut member is in engagement with the helical track, ensuring a correlated movement of the nut assembly and the scale drum during both dose setting and dose delivery.

The dose setting mechanism may further comprise a dose dial button operable to set and/or adjust a dose, e.g. by rotation about the longitudinal axis. The dose dial button may comprise a first engagement structure, such as e.g. a hook member or a toothed rim, and the nut connector may comprise a mating second engagement structure allowing releasable rotational locking to the first engagement structure.

The first engagement structure and the mating second engagement structure may be configured to be engaged when the first nut member and the second nut member take up the second relative axial position and to be disengaged when the first nut member and the second nut member take up the first relative axial position. In that case when the first nut member and the second nut member take up the second relative axial position an operation of the dose dial button will displace the scale drum relative to the housing while the entire nut assembly remains immobile on the guide structure, and when the first nut member and the second nut member take up the first relative axial position, during dose setting or dose delivery, an operation of the dose dial button will not have any effect on the scale drum, i.a. preventing any undesirable adjustment of the set dose during a dose administration.

In particular embodiments of the invention the variable volume reservoir is a cartridge type container having a generally cylindrical wall being closed at one end by a penetrable self-sealing septum and accommodating a displaceable sealing piston. The cartridge holds a drug substance in a chamber bounded by the cylindrical wall, the self-sealing septum, and the piston.

In the present context the term “bias member” is to be understood as any structure which is capable of providing a biasing force between two components, such as e.g. a coil spring, a leaf spring, an air spring, a foam rubber pad, etc.

In the present specification, reference to a certain aspect or a certain embodiment (e.g. “an aspect”, “a first aspect”, “one embodiment”, “an exemplary embodiment”, or the like) signifies that a particular feature, structure, or characteristic described in connection with the respective aspect or embodiment is included in, or inherent of, at least that one aspect or embodiment of the invention, but not necessarily in/of all aspects or embodiments of the invention. It is emphasized, however, that any combination of the various features, structures and/or characteristics described in relation to the invention is encompassed by the invention unless expressly stated herein or clearly contradicted by context.

The use of any and all examples, or exemplary language (e.g., such as, etc.), in the text is intended to merely illuminate the invention and does not pose a limitation on the scope of the same, unless otherwise claimed. Further, no language or wording in the specification should be construed as indicating any non-claimed element as essential to the practice of the invention.

### BRIEF DESCRIPTION OF THE DRAWINGS

In the following the invention will be further described with references to the drawings, wherein

FIG. 1 is an exploded view of a drug delivery device according to an embodiment of the invention,

FIG. 2 is an exploded view of a nut assembly used in the drug delivery device,

FIG. 3 is a longitudinal section view of the drug delivery device,

FIG. 4 is a close-up section view of a proximal portion of the drug delivery device as delimited by the area Q in FIG. 3,

FIG. 5 is a perspective longitudinal section view of the nut assembly in a locked state corresponding to the state of the drug delivery device shown in FIG. 4,

FIG. 6 is a close-up section view of a proximal portion of the drug delivery device showing the nut assembly in an unlocked state,

FIG. 7 is a close-up section view of a proximal portion of the drug delivery device showing the nut assembly during drug delivery, and

FIG. 8 is a perspective longitudinal section view of the nut assembly in a position corresponding to the one shown in FIG. 7.

In the figures like structures are mainly identified by like reference numerals.

### DESCRIPTION OF EXEMPLARY EMBODIMENTS

When in the following relative expressions, such as “upwards” and “downwards”, are used, these refer to the appended figures and not necessarily to an actual situation of use. The shown figures are schematic representations for which reason the configuration of the different structures as well as their relative dimensions are intended to serve illustrative purposes only.

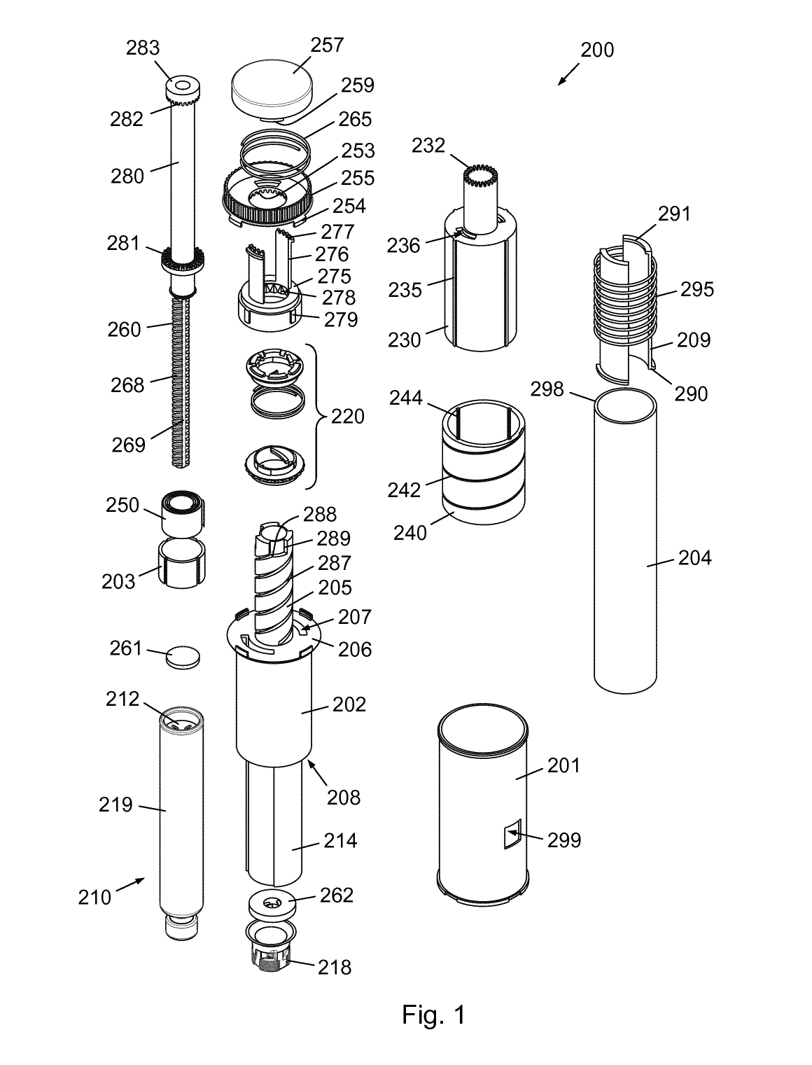

FIG. 1 is an exploded view of a drug delivery device according to an embodiment of the present invention. The drug delivery device is in the form of an automatic injection device 200 which is adapted to deliver set doses of drug from a cartridge 210. The injection device 200 comprises a proximal housing part 201 and a distal housing part 202 which are connected to form a unitary exterior cabinet of a generally tubular configuration. The distal housing part 202 has a distally extending cartridge holder 214 configured to hold and protect the cartridge 210, and to which a needle mount 218 for receiving an needle assembly (not shown) is attached, a transversal partition 206, and a proximally extending hollow spindle 205 serving as a guide structure for a nut assembly 220.

The spindle 205 comprises an exterior helical track 287 which leads into a longitudinal track 289 at a transition point 288. The helical track 287 is configured to be non-self-locking which means that its pitch enables a helical displacement of a mating nut structure under the influence of a purely axial external force. The transversal partition 206 has a pair of narrow slots 207, each slot 207 being configured to receive and to allow longitudinal displacement of a respective loading rod 209. Each loading rod 209 has a distal abutment edge 290 for interaction with a rim 298 of a protective cap 204 when the cap 204 is inserted into a cap receiving portion 208 of the distal housing part 202, and a proximal catch portion 291 for interaction with the nut assembly 220 in a manner that will be described in more detail in the following. A reset spring 295 is arranged to act between the distal abutment edge 290 and a distally facing portion of the transversal partition 206.

The nut assembly 220 is connected to a nut connector 275 which is an intermediate coupling element in a dose setting mechanism of the injection device 200. The nut connector 275 has an annular base from which two connecting arms 276 extend proximally. Each connecting arm 276 is provided with a toothing 277 at its free end. The base of the nut connector 275 has a circumferential set of teeth 278 on an interior surface portion and a plurality of protrusions 279 distributed along an exterior surface portion.

The toothing 277 on each arm 276 is adapted for rotational interlocking engagement with a mating toothed rim 253 of a dose dial 255. The dose dial 255 is connected to the proximal housing part 201 via a number of snap locks 254 and is configured to accommodate an injection button 257 in a way as to allow the injection button 257 to move axially up and down under influence of a button spring 265. The injection button 257 has a distally directed protrusion 259 for interaction with the nut connector 275 in a manner which will be further described in the below.

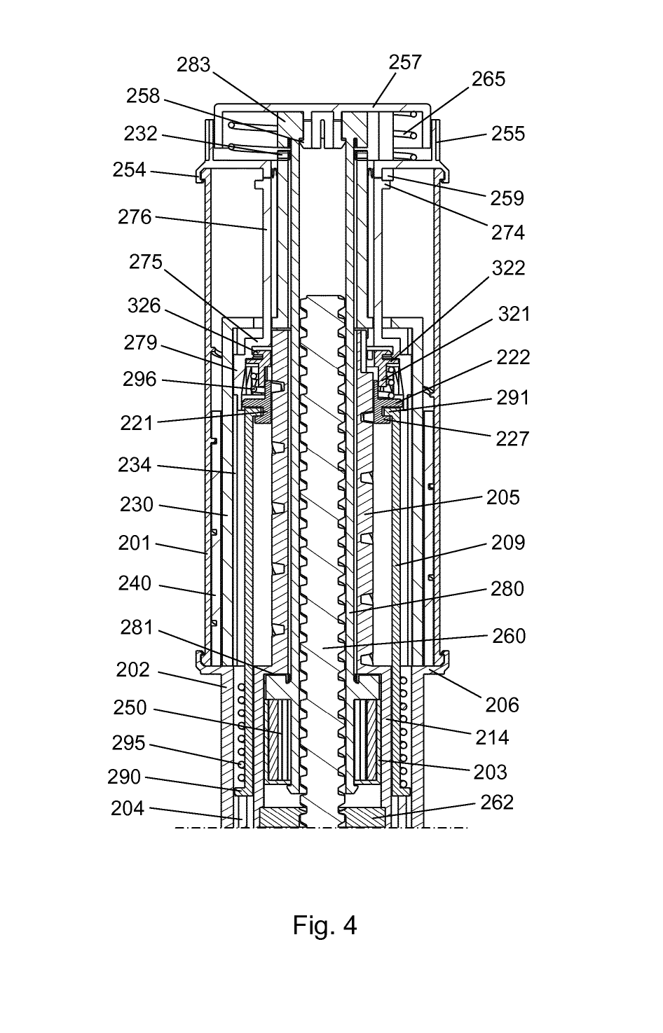

The protrusions 279 are received in respective longitudinal tracks 234 (see FIG. 4) in an interior surface of a scale drum connector 230, providing for an axially free but rotationally interlocked relation between the nut connector 275 and the scale drum connector 230. Openings 236 are provided in a transversal portion of the scale drum connector 230 to allow passage of the arms 276, and a toothed rim 232 is provided at the proximal end. On the exterior surface of the scale drum connector 230 a plurality of splines 235 are distributed which are in engagement with respective splines 244 in a scale drum 240.

The scale drum 240 is a tubular structure having a helically extending groove 242 for threaded engagement with an interior wall portion of the proximal housing part 201 as well as a plurality of dose related numerals (not shown) printed in a helical path on its exterior surface. The scale drum 240 is thus rotationally locked to the scale drum connector 230 and bound to move helically with respect to the proximal housing part 201 in response to a rotation of the scale drum connector 230. In every possible angular position of the scale drum 240 relative to the proximal housing part 201 at least one dose related numeral will be viewable through a window 299, allowing the user to easily identify the size of a set dose.

A driver 280 for effecting the dose delivery is slidingly arranged in the hollow of the spindle 205. The driver 280 has an elongated tubular body with a driver head 283 at a proximal end and a toothed transversal surface 281 arranged at a distal position. The driver head 283 has a distally oriented toothed rim 282 for rotational interlocking engagement with the toothed rim 232 during dose delivery, and the toothed transversal surface 281 is adapted to releasably engage with a mating toothing (not visible) on a distally facing portion of the partition 206, providing for a releasable rotational interlocking connection between the driver 280 and the distal housing portion 202.

The distal most portion of the driver 280 is connected to an inner free end of a spiral spring 250. An outer free end of the spiral spring 250 is connected to an interior surface of a spring housing 203, which spring housing 203 is rotationally locked in the distal housing part 202. The spiral spring 250 is pre-strained and has sufficient capacity to cause a complete emptying of the cartridge 210.

The driver 280 accommodates a piston rod 260 which has a thread 268 for interaction with a guide nut 262 that is fixedly arranged in the distal housing part 202. The thread 268 is interrupted by a longitudinal track 269 extending along the entire length of the piston rod 260. This provides for a splined connection between a protrusion (not visible) on an inner surface portion of the driver 280 and the longitudinal track 269, which ensures that any angular displacement of the driver 280 is passed on to the piston rod 260.

A distal end of the piston rod 260 abuts a piston washer 261 which again abuts a proximal surface portion of an axially displaceable piston 212 arranged in sealing connection with a cartridge wall 219. A counter-clockwise rotation of the driver 280, initiated for dose delivery, will thus cause a counter-clockwise rotation of the piston rod 260 which due to the threaded engagement with the guide nut 262 will be advanced helically downwards, pressing the piston washer 261 and the piston 212 into the cartridge 210 for expelling of a volume of drug through an injection needle (not shown) of a mounted needle assembly.

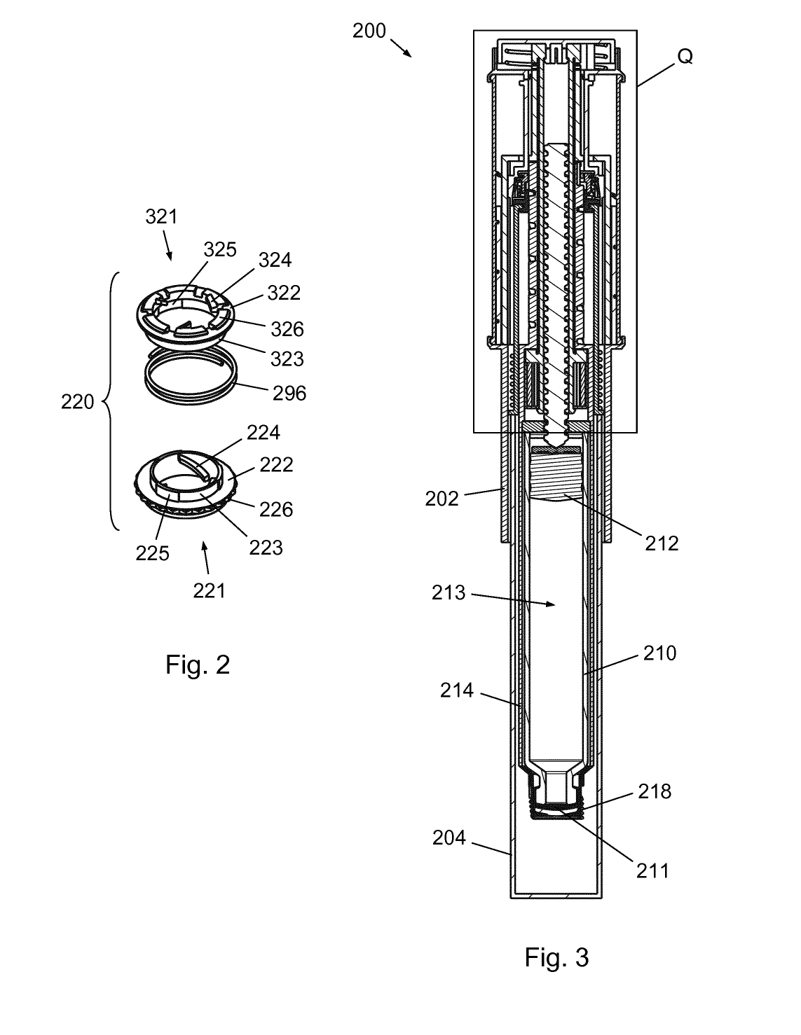

FIG. 2 is an exploded view detailing the nut assembly 220. The nut assembly 220 comprises a primary nut 221, a lock nut 321, and a nut spring 296. The primary nut 221 comprises a transversal spring base 222 which is provided with a round-going set of teeth 226 at its periphery, and a cylindrical portion 223 which has an inwardly projecting helical segment 224 for mating engagement with the helical track 287 and a plurality of circumferentially distributed exterior protuberances 225. The set of teeth 226 is configured to enable releasable engagement with the set of teeth 278, to provide for a rotational interlocking connection between the primary nut 221 and the nut connector 275 in a first relative axial position of the two and a rotational decoupling in a second relative axial position of the two.

The lock nut 321 comprises an annular base 322, from which a number of hook members 326 project, and a cylindrical portion 323 which has a plurality of indentations 325 distributed along an inner surface. Each indentation 325 is configured for sliding reception of a protuberance 225 to provide a rotational interlocking connection between the primary nut 221 and the lock nut 321. An inwardly directed protrusion 324 on the lock nut 321 is configured for mating engagement with the helical track 287 in one state of the nut assembly 220 and for engagement with the longitudinal track 289 in another state of the nut assembly 220. The hook members 326 are configured to axially retain an interior edge of the nut connector 275 and thereby prevent relative axial movement, while allowing relative rotation, between the nut connector 275 and the lock nut 321. The nut spring 296 is arranged to act between the spring base 222 and the annular base 322, biasing the primary nut 221 and the lock nut 321 axially away from one another.

FIG. 3 is a longitudinal section view of the injection device 200 in a dose setting state before a needle assembly has been mounted on the needle mount 218. The cap 204 is removably mounted on the injection device 200 to cover and protect the cartridge 210. The cartridge 210 holds a drug substance (not visible) in a variable volume chamber 213 which is bounded by the cartridge wall 219, the slidable piston 212, and a penetrable self-sealing septum 211.

FIG. 4 is a close-up view of a proximal portion of the injection device 200 delimited by the area Q in FIG. 3. The figure shows details of the various components and their respective connections with other components in the dose setting state of the injection device 200. In particular, the figure shows the nut assembly 220 in its axially expanded state, being immobilised on the spindle 205, i.e. where the protrusion 324 is in engagement with the longitudinal track 289, and where the primary nut 221 and the lock nut 321 are axially spaced apart by the nut spring 296. This can be termed a top position, or a dose prepared position, of the nut assembly 220. Notably, after removal of the cap 204 the nut assembly 220 will remain in the top position against the biasing axial force from the reset spring 295 acting on the primary nut 221 via the loading rods 209, which are axially fixed between the spring base 222 and a lower rim 227, because the lock nut 321 is prevented from rotating relative to the spindle 205 due to the engagement with the longitudinal track 289.

The injection button 257 is in its non-activated position relative to the proximal housing part 201, which means that the protrusion 259 rests against a rim 274 at the proximal end of one of the arms 276 without applying any significant force thereto, and the toothing 277 is in engagement with the toothed rim 253. A rotation of the dose dial 255 will thus cause a rotation of the nut connector 275 which due to the rotational interaction between the protrusions 279 and the longitudinal tracks 234 will cause a rotation of the scale drum connector 230. The rotation of the scale drum connector 230 is then transferred to the scale drum 240 due to the splined relationship between the two. Hence, when the nut assembly 220 is in the top position any angular displacement of the dose dial 255 will lead to a similar angular displacement of the scale drum 240. The angular displacement of the scale drum 240 relative to the proximal housing part 201 from a zero dose indicating position correlates directly with the size of the set dose which can be read through the window 299.

The injection button 257 is biased towards the non-activated position by the button spring 265 and the driver 280 is accordingly biased towards a proximal position due to the driver head 283 being axially retained by a retainer member 258. Thus, in the non-activated position of the injection button 257 the toothed transversal surface 281 is held firmly in rotational interlocking connection with the partition 206, and the spring 250 is thereby safely cocked.

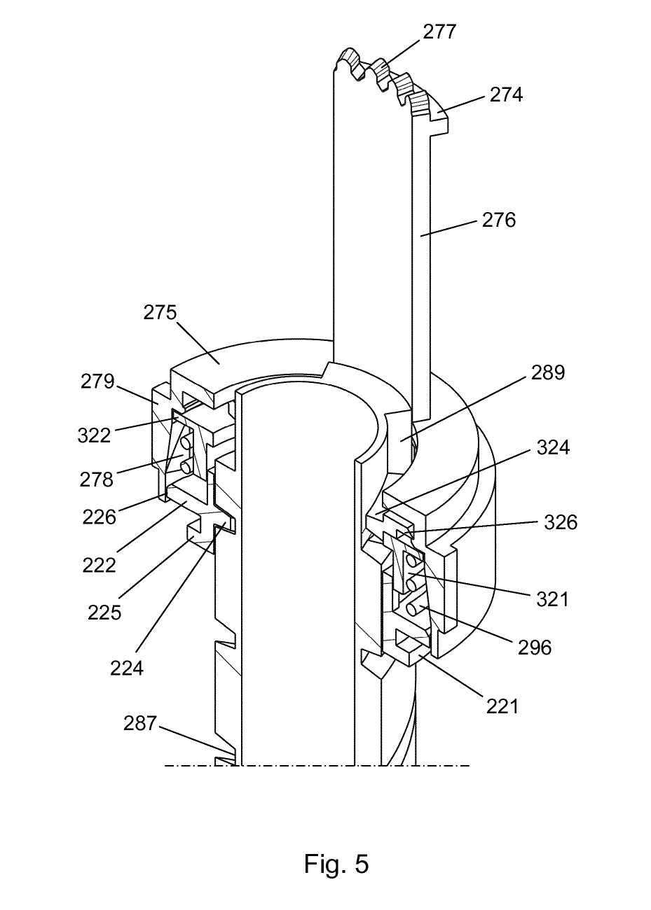

FIG. 5 is a perspective section view of a proximal portion of the spindle 205 showing the nut assembly 220 in the top position. Here, it can be seen more clearly that the helical segment 224 is in engagement with the helical track 287, while the protrusion 324 is in engagement with the longitudinal track 289, effectively preventing any rotation of the lock nut 321 and any movement at all of the primary nut 221 due to the engagement between the respective protuberances 225 and indentations 325 providing the rotational interlocking connection between the primary nut 221 and the lock nut 321. Further, it is seen that the relative axial position of the primary nut 221 and the nut connector 275 is such that the set of teeth 226 on the spring base 222 is disengaged from the set of teeth 278 on the interior surface of the base of the nut connector 275, whereby the nut connector 275 is capable of rotation with respect to the nut assembly 220.

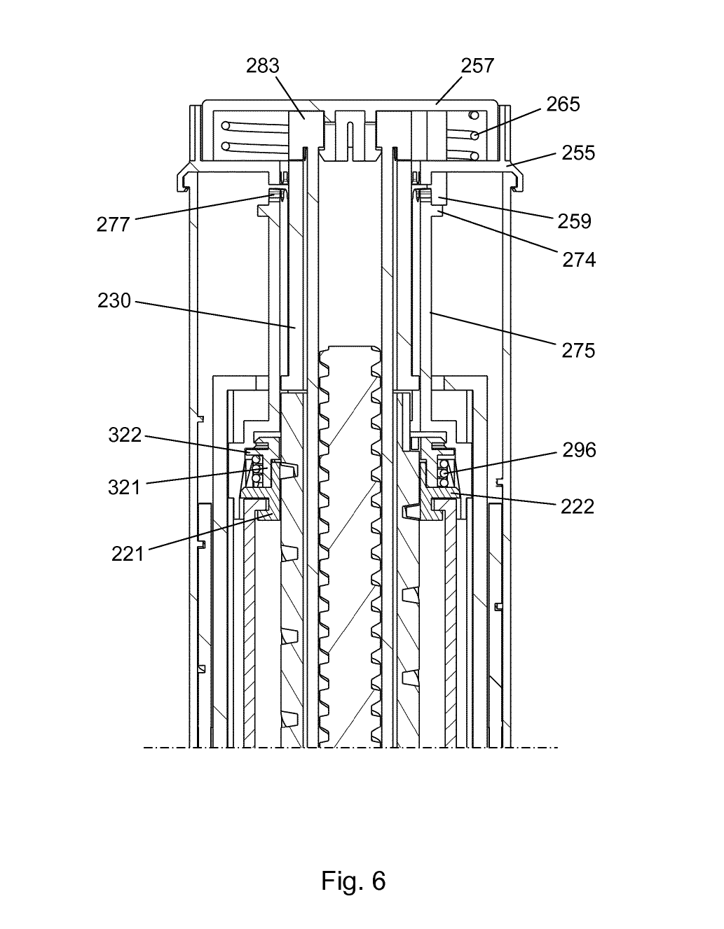

FIG. 6 shows a proximal portion of the injection device 200 and illustrates what happens when the injection button 257 is being depressed. Apart from a compression of the button spring 265 a depression of the injection button 257 entails four major changes to the interrelations between certain components. Firstly, the rim 274 is forced downwards by the protrusion 259 causing the toothing 277 to disengage from the toothed rim 253. The dose setting mechanism is thereby disabled because of the resulting decoupling of the scale drum 240 from the dose dial 205. Secondly, the lock nut 321 is forced downwards by the nut connector 275 exerting a pushing force on the annular base 322. This compresses the nut spring 296 and brings the lock nut 321 to the transition point 288. Thirdly, the driver head 283 is forced downwards, whereby the toothed rim 282 is brought into engagement with the toothed rim 232 on the scale drum connector 230, rotationally coupling the driver 280 and the scale drum 240. Fourthly, the downward movement of the driver 280 brings the toothed transversal surface 281 out of the rotational locking engagement with the partition 206, and the spring 250 is thus released.

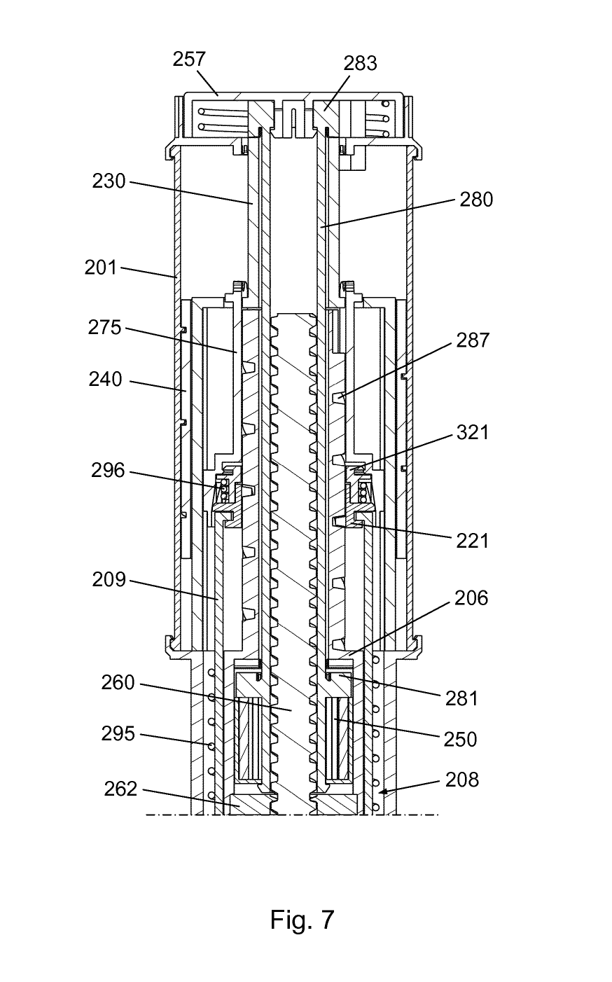

FIG. 7 shows the effect of the release of the spring 250. When the toothed transversal surface 281 is no longer prevented from angular displacement relative to the partition 206 the spring 250 is free to release stored energy for rotation of the driver 280. This causes a corresponding rotation of the piston rod 260 which due to the threaded engagement with the guide nut 262 is advanced helically through the distal housing part 202, forcing the piston 212 along the cartridge wall 219. As the driver head 283 rotates the scale drum connector 230 and the scale drum 240 rotate, which causes a helical upwards movement of the scale drum 240 in the proximal housing part 201.

The spring 250 will release energy and rotate the dose delivery components until the scale drum 240 reaches a physical stop surface in the proximal housing part 201 signifying an end-of-dose state of the injection device 200. The total angular displacement of the scale drum 240 relative to the proximal housing part 201 during the release of energy from the spring 250 correlates with the delivered amount of drug substance from the chamber 213, and in the end-of-dose state the scale drum 240 is in a zero dose indicating position. A subsequent removal of the depressing force from the injection button 257 will cause the button spring 265 to return the injection button 257 to the non-activated position, bringing along the driver head 283 and thereby decoupling the toothed rim 282 from the toothed rim 232 and moving the toothed transversal surface 281 back into rotational interlocking engagement with the partition 206.

Returning to the effect of the release of the spring 250, when the lock nut 321 is positioned at the transition point 288 the set of teeth 278 on the interior surface of the nut connector 275 has moved into engagement with the set of teeth 226 on the circumference of the spring base 222, and the nut connector 275 is thereby rotationally locked to the primary nut 221.

The spring induced rotation of the driver 280 which is transferred to the scale drum connector 230 is thus also transferred to the nut connector 275, due to the engagement between the respective protrusions 279 and longitudinal tracks 234, and to the nut assembly 220. Accordingly, the protrusion 324 enters into the helical track 287 and the nut assembly 220 as a unit is displaced helically down the spindle 205 since now both the primary nut 221 and the lock nut 321 are in engagement with the helical track 287. This helical displacement of the nut assembly 220 continues as long as the driver 280 rotates. As the primary nut 221 is thereby moved down towards the partition 206, bringing along the loading rods 209, the reset spring 295 is allowed to expand.

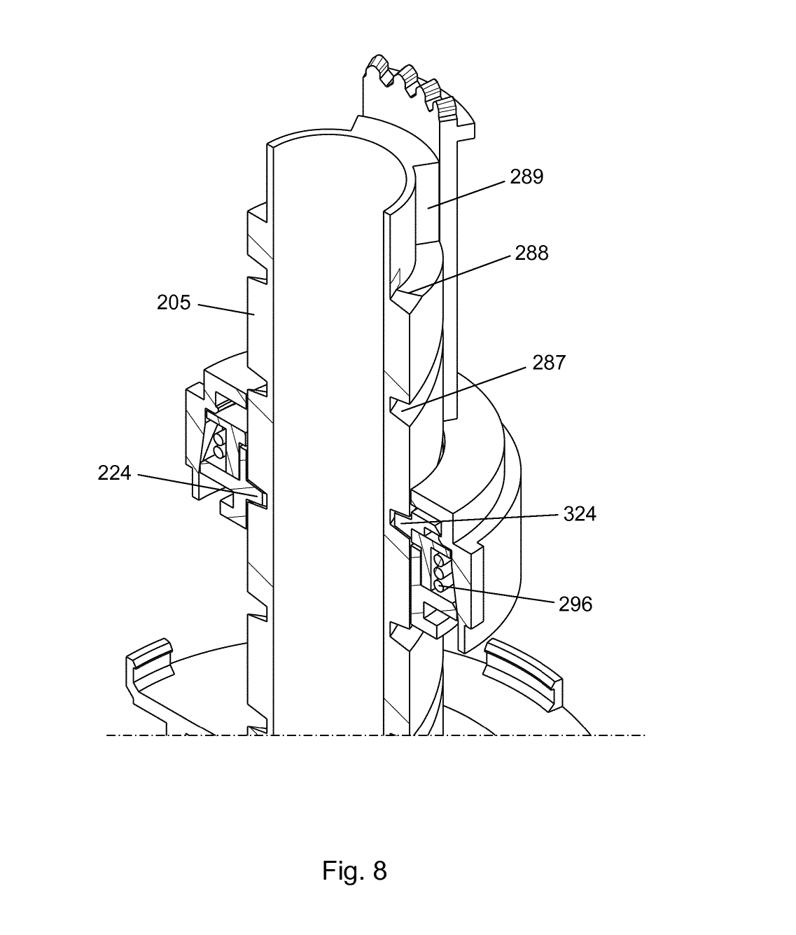

FIG. 8 shows a perspective section view of the nut assembly 220 in the same position as the one depicted by FIG. 7. The figure shows the nut assembly 220 in its axially compressed state, and it is clearly seen that both the helical segment 224 and the protrusion 324 are in engagement with the longitudinal track 287.

Following a completed dose delivery when the user re-mounts the cap 204 onto the injection device 200 the rim 298 of the cap 204 abuts the abutment edge 290 on the respective loading rods 209 and forces the loading rods 209 towards the proximal end of the proximal housing part 201 against the biasing force from the reset spring 295. Thereby, the respective catch portions 291 act on the spring base 222 and push the primary nut 221 in the same direction, while the reset spring 295 is compressed. Due to the helical segment 224 being in engagement with the helical track 287 and the rotational interlocking connection between the respective protuberances 225 and indentations 325 the entire nut assembly 220, in its compressed state, travels helically up the spindle 205 towards the transition point 288.

The resulting angular displacement of the primary nut 221 is transferred to the nut connector 275 and further on to the scale drum connector 230 and the scale drum 240, causing the scale drum 240 to move helically in the proximal housing part 201 away from the zero dose indicating position for automatic setting of a dose which corresponds to the dose that was just delivered. If during the travel of the nut assembly 220 up the spindle 205 the user suddenly chooses to remove the cap 204 the reset spring 295 will expand and drive the nut assembly 220 back down the spindle 205, causing an opposite angular displacement of the primary nut 221, the nut connector 275, the scale drum connector 230, and the scale drum 240, which will take the scale drum 240 back to the zero dose indicating position. The reset spring 295 thus serves to ensure that the scale drum 240 is not left in an intermediate position where an arbitrary dose is set.

When the lock nut 321 passes the transition point 288 the nut spring 296 expands and forces the protrusion 324 further proximally along the longitudinal track 289 while the primary nut 221 remains in position because the cap 204 is then fully mounted on the injection device 200. The nut assembly 220 is thereby immobilised on the spindle 205 and the reset spring 295 is securely cocked. Further, the axial displacement of the lock nut 321 causes the nut connector 275 to re-engage with the dose dial 255, thereby allowing for manual adjustment of the automatically set dose (compare FIG. 7 and FIG. 4).

1. A medical device comprising:
a housing,
a guide structure arranged stationarily with respect to the housing and extending along a longitudinal axis, the guide structure comprising a helical track and a longitudinal track being connected with the helical track at a transition point, and
a nut assembly adapted to move along the guide structure, the nut assembly comprising:
a first nut member arranged in engagement with the helical track,
a second nut member capable of engagement with the helical track and with the longitudinal track, the second nut member being rotationally locked to the first nut member, and
a bias member arranged to bias the first nut member and the second nut member away from one another, such that the first nut member and the second nut member take up a first relative axial position when they are both in engagement with the helical track and a second relative axial position when the second nut member is in engagement with the longitudinal track.

2. A medical device according to claim 1, further comprising:
a spring, and
a spring interfacing structure,
wherein the spring is arranged to act between a portion of the housing and the spring interfacing structure, and
wherein the spring interfacing structure is axially fixed with respect to the first nut member, the spring interfacing structure thereby being arranged to tension the spring when the first nut member moves towards the transition point.

3. A medical device according to claim 2, wherein the helical track is non-self-locking, and wherein the spring is a linear spring.

4. A medical device according to claim 3, further comprising a nut release structure comprising a nut return button displaceable relative to the housing from a first position to a second position, the nut release structure being coupled with the second nut member and configured to cause movement of the second nut member along the longitudinal track to the transition point in response to a displacement of the nut return button from the first position to the second position.

5. A medical device according to claim 4, further comprising:
a cap removably mountable onto the housing, and
a cap receiving portion for receiving the cap when the cap is being mounted onto the housing,
wherein the nut assembly is operatively coupled with the cap receiving portion and configured to move along the helical track in response to the cap being mounted onto the housing.

6. A medical device according to claim 5, wherein nut assembly is operatively coupled with the cap receiving portion via the spring interfacing structure, and
wherein during mounting of the cap onto the housing an edge of the cap is adapted to abut the spring interfacing structure and move the spring interfacing structure along the longitudinal axis.

7. A medical device according to claim 4, wherein the nut release structure further comprises a nut connector being axially locked relative to the second nut member, and
wherein the nut connector is further rotationally locked relative to the first nut member when the first nut member and the second nut member take up the first relative position, and rotationally decoupled from the first nut member when the first nut member and the second nut member take up the second relative position.

8. A medical device according to claim 7, further comprising:
a variable volume reservoir comprising a penetrable septum and being adapted to hold a liquid substance,
a dose setting mechanism for setting a dose to be delivered from the variable volume reservoir, the dose setting mechanism comprising a scale drum threadedly engaged with an interior portion of the housing, and
a dose delivery mechanism for delivering a set dose,
wherein the nut connector is rotationally locked relative to the scale drum.

9. A medical device according to claim 8, wherein the dose setting mechanism further comprises a dose dial button for selective setting of a dose, the dose dial button being capable of rotation about the longitudinal axis and comprising a first engagement structure,
wherein the nut connector comprises a mating second engagement structure for releasable rotational interlocking connection with the first engagement structure, and
wherein the first engagement structure and the mating second engagement structure are configured to be engaged when the first nut member and the second nut member take up the second relative axial position and disengaged when the first nut member and the second nut member take up the first relative axial position.
